(19) United States
(12) Patent Application Publication
(10) Pub. No.: US 2025/0295618 A1
(43) Pub. Date: Sep. 25, 2025

(54) PARASYMPATHETIC NERVE ACTIVATION AGENT AND PARASYMPATHETIC NERVE ACTIVATION COMPOSITION

(71) Applicant: MARUZEN PHARMACEUTICALS CO., LTD., Hiroshima (JP)

(72) Inventor: Hiroyuki KAYAKI, Hiroshima (JP)

(21) Appl. No.: 18/864,263

(22) PCT Filed: Apr. 17, 2023
(86) PCT No.: PCT/JP2023/015383

§ 371 Date: Nov. 8, 2024

(30) Foreign Application Priority Data
May 13, 2022 (JP) 2022-079699

(51) Int. Cl. A61K 31/192; A61P 25/02

(52) U.S. Cl. CPC A61K 31/192; A61P 25/02

(57) ABSTRACT

A parasympathetic nerve activation agent includes a compound represented by Structural Formula (1).

### TECHNICAL FIELD

The present invention relates to parasympathetic nerve activation agents and parasympathetic nerve activation compositions.

### BACKGROUND ART

In recent years, stress has become a major problem. Stimuli externally applied to the mind and body are called stressors. Physical stressors, such as weather, temperature change, noise, and the like, chemical stressors, such as drugs, chemical substances that harm the body, and the like, and psychological and social stressors, such as human relationship, work-related problems, household problems, and the like, are known.

In order to maintain the internal environment constant against external stimuli, the system known as a autonomic nervous system is in a living body. The autonomic nerves include parasympathetic nerves and parasympathetic nerves.

The parasympathetic nerves are stimulated when the body is relaxed, such as during eating or during sleep. As the parasympathetic nerves are stimulated, the parasympathetic nerves act to lower heart rate, expand blood vessels, activate gastrointestinal functions, or the like. The activation of the parasympathetic nerves is expected to have an effect of relaxing or calming a tensed state of the sympathetic nerves.

As a substance for activating parasympathetic nerves, thyme that is an herb, which is native to the Mediterranean coast in southern Europe, has been reported (see, for example, Patent Document 1).

However, there is still a strong demand for a novel material that has an effect on parasympathetic activation, and is highly safe, and therefore can be widely used as a substance for food and drink products, pharmaceutical products, research reagents, and the like.

Note that, the name of the compound represented by Structural Formula (1) below is 3-(4-hydroxy-3-methoxyphenyl)propionic acid. It has been known that the compound represented by Structural Formula (1) below can be detected when a certain type of lactic acid bacterium is cultured in a medium including a specific substance (see, for example, Patent Document 2).

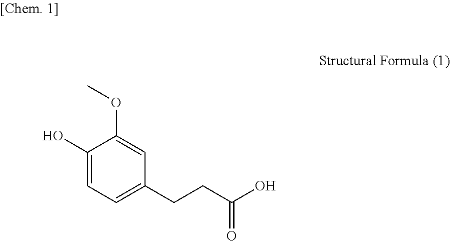

The compound represented by Structural Formula (1) is an active ingredient of a dipeptidyl peptidase IV activity inhibitor, and is known to be formulated in drink and food products for inhibiting dipeptidyl peptidase IV activity (see, for example, Patent Document 3). However, it has not been known that the compound represented by Structural Formula (1) has a parasympathetic activation effect.

### CITATION LIST

### Patent Documents

Patent Document 1: Japanese Unexamined Patent Application Publication No. 2022-007470

Patent Document 2: Japanese Unexamined Patent Application Publication No. 2014-003929

Patent Document 3: Japanese Unexamined Patent Application Publication No. 2020-055887

### SUMMARY OF INVENTION

### Technical Problem

The present inventors aim to solve the various problems existing in the related art, and achieve the following object. Specifically, the object of the present invention is to provide a parasympathetic nerve activation agent and a parasympathetic nerve activation composition, which have an excellent parasympathetic activation effect and are highly safe.

Solution to Problem

The present inventors have conducted research to solve the above problems. As a result, the present inventors have reached the insight such that a compound represented by Structural Formula (1) below has an excellent parasympathetic activation effect, is highly safe, and is effective for use associated with parasympathetic activation.

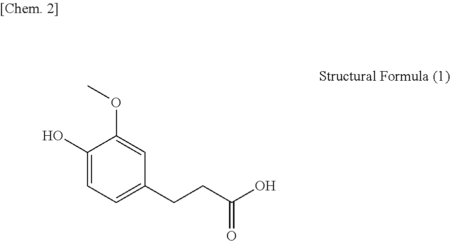

The present invention is based on the above insight of the present inventors, and the means for solving the above problems are as follows.
<1>A parasympathetic nerve activation agent including a compound represented by Structural Formula (1) below.

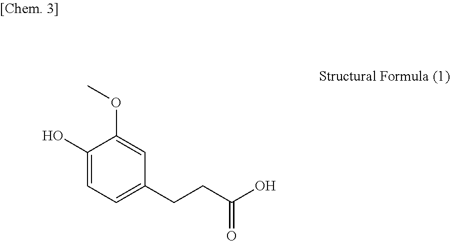

<2>The parasympathetic nerve activation agent according to <1>, in which the parasympathetic nerve is a parasympathetic nerve innervating at least one selected from the group consisting of stomach, pancreas, and intestines.
<3>A parasympathetic nerve activation composition including the parasympathetic nerve activation agent according to <1>or <2>.

### Effects of Invention

According to the parasympathetic nerve activation agent and parasympathetic nerve activation composition of the present invention, the various problems existing in the related art can be solved, the above object can be achieved, and a parasympathetic nerve activation agent and a parasympathetic nerve activation composition, which have an excellent parasympathetic activation effect and are highly safe, can be provided.

### BRIEF DESCRIPTION OF THE DRAWINGS

FIG. 1 is a diagram illustrating a change in the neural activity of distal branches of vagus (parasympathetic) nerve for the stomach in Test Example 1.

FIG. 2 is a diagram illustrating a change in the neural activity of distal branches of vagus (parasympathetic) nerve for the pancreas.

FIG. 3 is a diagram illustrating a change in the neural activity of distal branches of vagus (parasympathetic) nerve for the intestines.

### DESCRIPTION OF EMBODIMENTS

### Parasympathetic Nerve Activation Agent

The parasympathetic nerve activation agent of the present invention includes a compound represented by Structural Formula (1) below as an active ingredient, and may further include other ingredients as necessary.

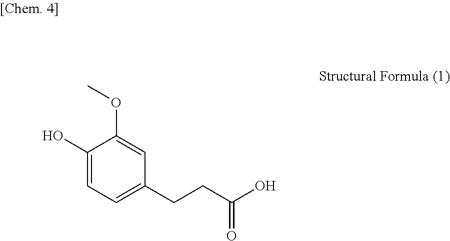

In the present specification, the parasympathetic activation means that an activity level of parasympathetic nerves is increased compared with a state before administering the parasympathetic nerve activation agent.

The parasympathetic nerve activation agent preferably activates the parasympathetic nerve innervating at least one selected from the group consisting of the stomach, the pancreas, and the intestines.

The fact that the compound represented by Structural Formula (1) has a parasympathetic activation effect and is effective as a parasympathetic nerve activation agent has not been known at all in the related art and is a novel insight of the present inventors.

### Compound Represented by Structural Formula (1)

The name of the compound represented by Structural Formula (1) below is 3-(4-hydroxy-3-methoxyphenyl)propionic acid) (may be referred to as “HMPA” hereinafter).

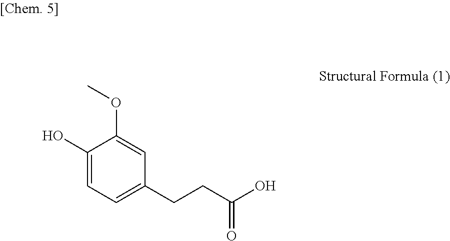

The compound represented by Structural Formula (1) is a known compound. As the compound represented by Structural

Formula (1), a commercially available product may be used, or a compound extracted from plants or the like may be used.

An amount of the compound represented by Structural Formula (1) in the parasympathetic nerve activation agent is not particularly limited, and may be appropriately selected.

The parasympathetic nerve activation agent may be composed solely of the compound represented by Structural Formula (1), or may be obtained by formulating the compound represented by Structural Formula (1) as a preparation.

### Other Ingredients

The above other ingredients are not particularly limited, as long as the effects obtainable by the present invention are not adversely affected. The other ingredients are appropriately selected according to a usage form of the parasympathetic nerve activation agent. Examples of the other ingredients include excipients, moisture-proof agents, preservatives, enhancers, thickeners, emulsifiers, antioxidants, sweeteners, acidulants, seasonings, coloring agents, fragrances, whitening agents, humectants, oil components, UV absorbers, surfactants, alcohols, powder components, colorants, aqueous components, water, skin nutrients, and the like. The above other ingredients may be used alone or in combination.

An amount of the other ingredients in the parasympathetic nerve activation agent is not particularly limited, and may be appropriately selected.

The parasympathetic nerve activation agent can be formulated into a desired dosage form, such as powders, granules, tablets, liquids, or the like, using a pharmaceutically acceptable carrier, such as dextrin, cyclodextrin, or the like, or any other auxiliary agents according to a common method. As the auxiliary agents, for example, excipients, binders, disintegrating agents, lubricants, stabilizers, flavoring agents, or the like can be used.

The parasympathetic nerve activation agent can be used by blended in another composition (e.g., the below-described parasympathetic nerve activation composition), and can be also used as a parenteral administration agent, such as injection, drip, suppository, and the like, or an ointment, an eye drop, a fluid preparation for external application, a patch, or the like.

Note that, the parasympathetic nerve activation agent may use, as necessary, another component having a parasympathetic activation effect blended together with the compound represented by Structural Formula (1) as active ingredients.

### Use

Use of the parasympathetic nerve activation agent is not particularly limited, and may be appropriately selected. Examples of the use include a variety of use, such as pharmaceutical products, quasi-drugs, food and drink products, and the like.

Since the parasympathetic nerve activation agent has an excellent parasympathetic activation effect and is highly safe, the parasympathetic nerve activation agent can be suitably used, for example, as an active ingredient of the below-described parasympathetic nerve activation composition.

The parasympathetic nerve activation agent is suitably applied to humans, but can be applied to animals other than humans (e.g., mice, rats, hamsters, dogs, cats, cows, pigs, monkeys, etc.) as long as the functions and effects of the parasympathetic nerve activation agent can be exhibited.

The parasympathetic nerve activation agent is suitably used for a subject whose activity level of the parasympathetic nerves is lowered.

Usage of the parasympathetic nerve activation agent is not particularly limited, and may be appropriately selected. Examples of the usage include oral administration, parenteral administration, external application, and the like.

A dosage form of the parasympathetic nerve activation agent is not particularly limited, and may be appropriately selected from dosage forms known in the related art according to the intended purpose.

A production method for the parasympathetic nerve activation agent in any dosage form is not particularly limited, and may be appropriately selected from methods known in the related art.

An administration method, an administration dose, an administration site, an administration period, an administration interval, and the like of the parasympathetic nerve activation agent are not particularly limited, and may be appropriately selected.

For example, as an example of the administration dose of the parasympathetic nerve activation agent in a human, the administration dose for a body weight of 60 kg per day, which is converted into the amount of the compound represented by Structural Formula (1), is from 0.005 mg to 25 mg, or the like.

Moreover, as an example of the administration dose of the parasympathetic nerve activation agent in a human for enhancing the activity of the parasympathetic nerve innervating the stomach, the administration dose for a body weight of 60 kg per day, which is converted into the amount of the compound represented by Structural Formula (1), is from 0.5 mg to 25 mg, or the like.

Moreover, as an example of the administration dose of the parasympathetic nerve activation agent in a human for enhancing the activity of the parasympathetic nerve innervating the pancreas, the administration dose for a body weight of 60 kg per day, which is converted into the amount of the compound represented by Structural Formula (1), is from 0.005 mg to 2 mg, or the like.

Moreover, as an example of the administration dose of the parasympathetic nerve activation agent in a human for enhancing the activity of the parasympathetic nerve innervating the intestines (small intestine or large intestine), the administration dose for a body weight of 60 kg per day, which is converted into the amount of the compound represented by Structural Formula (1), is from 0.005 mg to 2 mg, or the like.

Moreover, the parasympathetic nerve activation agent of the present invention can be also used as a reagent for research associated with an action mechanism of the parasympathetic activation effect.

### Parasympathetic Nerve Activation Composition

The parasympathetic nerve activation composition of the present invention includes the parasympathetic nerve activation agent of the present invention, and may further include other ingredients as necessary.

### Parasympathetic Nerve Activation Agent

The parasympathetic nerve activation agent is the above parasympathetic nerve activation agent of the present invention.

An amount of the parasympathetic nerve activation agent in the parasympathetic nerve activation composition is not particularly limited, and may be appropriately adjusted according to a usage form of the parasympathetic nerve activation composition or the like. The amount of the parasympathetic nerve activation agent, which is converted into the amount of the compound represented by Structural Formula (1), is preferably from 0.0001% by mass to 20% by mass, and more preferably from 0.0001% by mass to 10% by mass. The parasympathetic nerve activation composition may be composed solely of the parasympathetic nerve activation agent.

### Other Ingredients

Other ingredients in the parasympathetic nerve activation composition are not particularly limited, and may be appropriately selected according to a usage form of the parasympathetic nerve activation composition. Examples of the above other ingredients include the same ingredients as the other ingredients described in the above section of the parasympathetic nerve activation agent. Such ingredients may be used alone or in combination.

An amount of the other ingredients in the parasympathetic nerve activation composition is not particularly limited, and may be appropriately selected.

### Embodiments

Embodiments of the parasympathetic nerve activation composition are not particularly limited, and may be appropriately selected. Examples of the embodiments include pharmaceutical products, quasi-drugs, drink and food products, and the like.

The parasympathetic nerve activation composition can be used daily, and can extremely effectively exhibit a variety of pharmacological activity including the parasympathetic activation effect due to the action of the compound represented by Structural Formula (1) serving as an active ingredient.

The parasympathetic nerve activation composition is suitably applied to humans, but can be applied to animals other than humans (e.g., mice, rats, hamsters, dogs, cats, cows, pigs, monkeys, etc.) as long as the functions and effects of the parasympathetic nerve activation composition can be exhibited.

The parasympathetic nerve activation composition is preferably used for a subject whose activity level of the parasympathetic nerves is lower than a normal level.

Usage of the parasympathetic nerve activation composition is not particularly limited, and may be appropriately selected. Examples of the usage include oral administration, parenteral administration, external application, and the like. The usage is preferably oral administration.

Examples of the composition for oral administration include oral preparations, food and drink products, and the like. In the present specification, the food and drink products refer to products that have little risk for harming human health and can be consumed in ordinary social life through oral administration or gastrointestinal administration. The food and drink products are not limited to categories according to the administrative classification, such as foods, pharmaceutical products, and quasi-drugs. Accordingly, the food and drink products include a wide range of food and drink products including general food products, health foods (functional food and drink products), health-promoting food products (food for specified health uses, food with nutrient function claims, and food with functional claims), quasi-drugs, pharmaceutical products, etc., which are for oral intake.

A type of the composition for the oral administration is not particularly limited, and may be appropriately selected. Examples thereof include: beverages such as tea beverages, soft drinks, carbonated beverages, nutritional beverages, fruit-based beverages, lactic beverages, alcoholic beverages, coffee beverages, coffee-containing soft drinks, and the like (including concentrates of the foregoing beverages and powders for preparing the foregoing beverages); frozen desserts such as ice cream, sherbet, shaved ice, and the like; noodles, such as buckwheat noodles, udon noodles, starch noodles, Gyoza skins,

Shumai skins, Chinese noodles, instant noodles, and the like; confectionary, such as hard candies, candies, chewing gums, chocolate, tablets, snacks, biscuits, jelly, jams, creams, sweet pastries, bread, and the like; fishery products, such as crabs, salmon, clams, tuna, sardine, shrimp, bonito, mackerel, whale, Oysters, Pacific saury, squid, Anadara broughtonii, scallops, abalone, sea urchin, salmon roe, Sulculus diversicolor supertexta, and the like; fish or meat processed foods, such as fish paste, ham, sausages, and the like; dairy products, such as processed milk, fermented milk, and the like; fat or oil, or fat or oil processed food products, such as salad oil, tempura oil, margarines, mayonnaise, shortening, whipping creams, dressing, and the like; seasonings, such as sauces and dipping sauces; retort pouch-packaged foods, such as curry, stew, Oyakodon, gruel, rice soups, Chukadon, a bowl of rice with breaded pork cutlets, a bowl of rice with tempura, Unadon, Hayashi rice, Oden, Mapo tofu, a bowl of rice with beef, Bolognese sauces, egg soup, omelette made with rice, Gyoza, Shumai, hamburgers, meat balls, and the like; daily dishes, such as salads and pickles; various forms of health, cosmetic, or nutritional supplementary foods; pharmaceutical products and quasi-drugs, such as tablets, powders, capsules, granules, extracts, syrups, drink preparations, lozenges, mouthwash, and the like; oral fresheners used inside the mouth, such as mouth freshener and breath freshener; toothpaste; and the like.

A production method for the parasympathetic nerve activation composition is not particularly limited and may be appropriately selected according to a usage form of the parasympathetic nerve activation composition.

A usage dose, a usage site, a usage period, a usage interval and the like of the parasympathetic nerve activation composition are not particularly limited, and may be appropriately selected.

For example, as an example of the usage dose of the parasympathetic nerve activation composition in a human, the usage dose for a body weight of 60 kg per day, which is converted into the amount of the compound represented by Structural Formula (1), is from 0.005 mg to 25 mg, or the like.

Moreover, as an example of the usage dose of the parasympathetic nerve activation composition in a human for enhancing the activity of the parasympathetic nerve innervating the stomach, the usage dose for a body weight of 60 kg per day, which is converted into the amount of the compound represented by Structural Formula (1), is from 0.5 mg to 25 mg, or the like.

Moreover, as an example of the usage dose of the parasympathetic nerve activation composition in a human for enhancing the activity of the parasympathetic nerve innervating the pancreas, the usage dose for a body weight of 60 kg per day, which is converted into the amount of the compound represented by Structural Formula (1), is from 0.005 mg to 2 mg, or the like.

Moreover, as an example of the usage dose of the parasympathetic nerve activation composition in a human for enhancing the activity of the parasympathetic nerve innervating the intestines (small intestine or large intestine), the usage dose for a body weight of 60 kg per day, which is converted into the amount of the compound represented by Structural Formula (1), is from 0.005 mg to 2 mg, or the like.

As described above, the parasympathetic nerve activation agent and parasympathetic nerve activation composition of the present invention have an excellent parasympathetic activation effect.

Accordingly, the present invention also relates to a method of activating parasympathetic nerves including administration of at least one selected from the group consisting of the parasympathetic nerve activation agent and the parasympathetic nerve activation composition to a subject.

In addition, as the activity of the parasympathetic nerve innervating the stomach is activated, gastrointestinal functions, secretion of gastric acid, and peristalsis of the stomach are facilitated, thereby enhancing digestive and absorptive functions.

As the activity level of the parasympathetic nerve innervating the pancreas is increased, release of digestive enzymes from the exocrine glands to the intestines is facilitated to promote digestive functions, and secretion of insulin from B cells of the islets of Langerhans, which are endocrine glands, is facilitated to achieve an anti-diabetic effect of lowering blood sugar levels.

As the activity level of the parasympathetic nerve innervating the intestine is increased, digestive and adsorptive capability of the intestine is enhanced, and the peristalsis action of the intestine is facilitated, thereby promoting the bowel movement.

Accordingly, the present invention also relates to a digestion promoter, a hypoglycemic agent, or a peristalsis promoter, which includes the compound represented by the structural formula (1). Moreover, the present invention relates to a digestion promotion composition including the digestion promoter, a hypoglycemic composition including the hypoglycemic agent, or a peristalsis promotion composition including the peristalsis promoter. In addition, the present invention relates to a digestion promoting method including administration of at least one selected from the group consisting of the digestion promoter and the digestion promotion composition to a subject, a blood sugar level reduction method including administration of at least one selected from the group consisting of the hypoglycemic agent or the hypoglycemic composition to a subject, or a peristalsis promoting method including administration of at least one selected from the group consisting of the peristalsis promoter and the peristalsis promotion composition to a subject.

### EXAMPLES

Test Example and Formulation Examples of the present invention will be described hereinafter, but the present invention is not limited to Text Example and Formulation Examples below.

### Test Example 1

The effect of the intragastric administration of the compound represented by Structural Formula (1) on the activity of the parasympathetic nerve innervating the stomach, the pancreas, or the intestine was tested in the following manner.

### Measurement

For the experiment, male Whistar rats (approximately 9 weeks old) each having a body weight of approximately 300 g were kept in a 24° C. thermostatic animal room for 1 week or longer with a 12-hour light-dark cycle (lighting from 8:00 to 20:00). On the day of the experiment, the rats were fasted for 3 hours, followed by anesthetizing with urethane. A cannula for intragastric administration was inserted into each of the rats, and then distal branches of the parasympathetic nerve (vagus nerve) innervating the stomach, the pancreas, or the intestines (small intestine and large intestine) were lifted with a silver electrode, and the electrical activity thereof was measured. When the measurement value stabilized (at approximately 13:00), an aqueous solution of the compound represented by Structural Formula (1) (produced by Tokyo Chemical industry Co., Ltd.) was intragastrically administered using the cannula at a dose of 0.015 mg/0.5 mL/300 g of body weight, 0.0015 mg/0.5 mL/300 g of body weight, or 0.00015 mg/0.5 mL/300 g of body weight, and the change in the neural activity of the distal branches of the above parasympathetic nerves was measured electrophysiologically for 60 minutes. In addition, as a control, water serving as a solvent was intragastrically administered using the cannula at a dose of 0.5 mL/300 g of body weight, and the change in the neural activity of the distal branches of the above parasympathetic nerves was measured electrophysiologically for 60 minutes.

From the start of the operation to the end of the measurement, a tube was inserted into the trachea to secure an airway, and the body temperature (rectal temperature of the rat) was maintained at 37.0±0.5° C. with a warming device.

The data of the neural activity was analyzed as the mean value of the firing rate per 5 seconds (pulse/5 s) every 5 minutes, and represented by a percentage relative to the mean value (a value at 0 minutes) at 5 minutes before the onset of stimulation being determined as 100%.

### Results

### Stomach

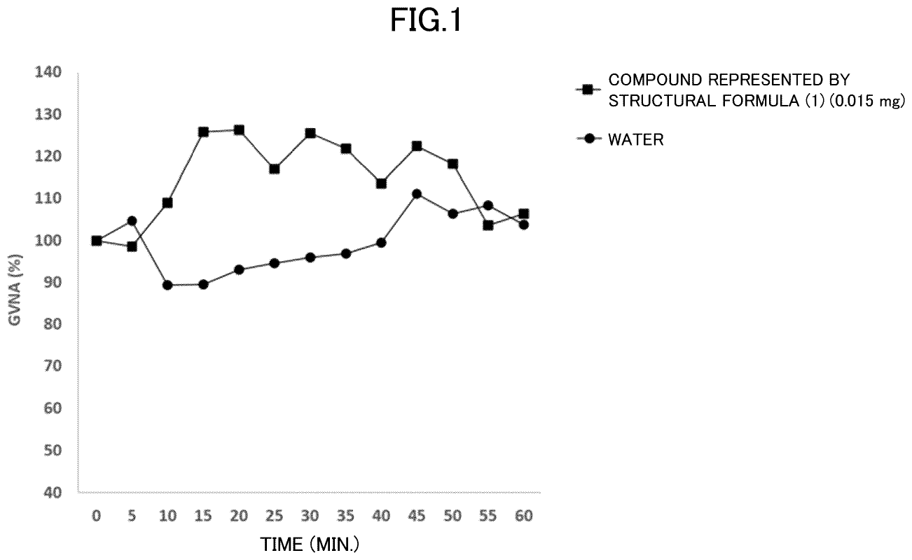

FIG. 1 illustrates, as a percentage with respect to the mean value (a value at 0 minutes) at 5 minutes before the onset of stimulation being determined as 100%, the result of the change in the neural activity of the distal branches of vagus (parasympathetic) nerve for the stomach (gastric vagal nerve activity, GVNA) when the aqueous solution including 0.015 mg of the compound represented by the structural formula (1) was intragastrically administered at a dose of 0.5 mL/300 g of body weight, or water serving as the solvent was intragastrically administered at a dose of 0.5 mL/300 g of body weight.

As presented in FIG. 1, it was made clear that the group to which the aqueous solution including 0.015 mg of the compound represented by Structural Formula (1) was administered at the dose of 0.5 mL/300 g of body weight (“ [symbol] ” in FIG. 1) had the GVNA value higher than the GVNA value of the group to which water serving as the solvent was administered at the dose of 0.5 mL/300 g of body weight (“●” in FIG. 1). It was found from the above results that the compound represented by the structural formula (1) had the effect of activating the activity of the parasympathetic nerve innervating the stomach.

As the activity level of the parasympathetic nerve innervating the stomach is increased, gastrointestinal functions, secretion of gastric acid, and peristalsis of the stomach are facilitated, thereby enhancing digestive and absorptive functions.

### Pancreas

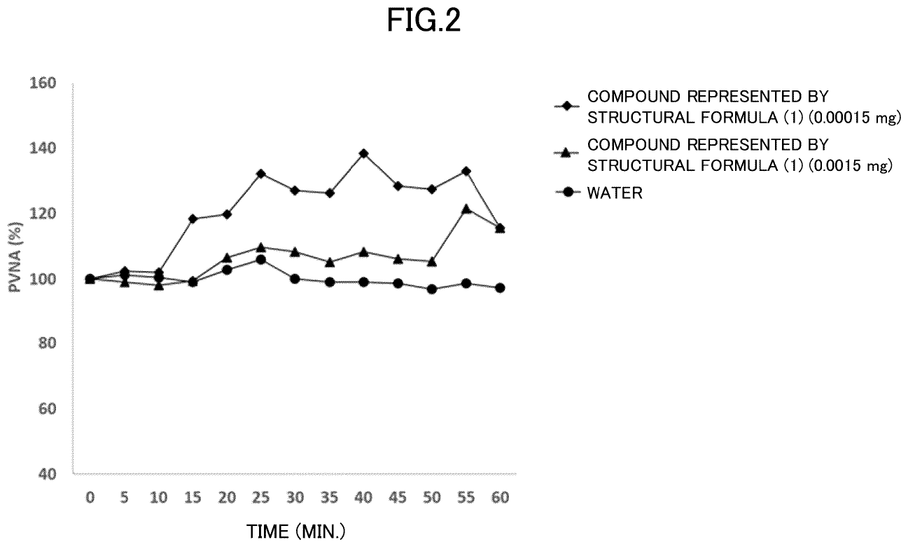

FIG. 2 illustrates the result of the change in the neural activity of the distal branches of vagus (parasympathetic) nerve for the pancreas (pancreatic vagal nerve activity, PVNA) when the aqueous solution including 0.0015 mg or 0.00015 mg of the compound represented by the structural formula (1) was intragastrically administered at a dose of 0.5 mL/300 g of body weight, or water serving as the solvent was intragastrically administered at a dose of 0.5 mL/300 g of body weight, as a percentage with respect to the mean value (a value at 0 minutes) of 5 minutes before the onset of stimulation being 100%.

As presented in FIG. 2, it was made clear that the group to which the aqueous solution including 0.0015 mg of the compound represented by Structural Formula (1) was administered at the dose of 0.5 mL/300 g of body weight (“ [symbol] ” in FIG. 2) and the group to which the aqueous solution including 0.00015 mg of the compound represented by Structural Formula (1) was administered at the dose of 0.5 mL/300 g of body weight (“♦” in FIG. 2) had the PVNA value higher than the PVNA value of the group to which water serving as the solvent was administered at the dose of 0.5 mL/300 g of body weight (“●” in FIG. 2). It was found from the above results that the compound represented by the structural formula (1) had the effect of activating the activity of the parasympathetic nerve innervating the pancreas.

As the activity level of the parasympathetic nerve innervating the pancreas is increased, release of digestive enzymes from the exocrine glands to the intestine is facilitated to promote digestive functions, and secretion of insulin from B cells of the islets of Langerhans, which are endocrine glands, is facilitated to achieve an anti-diabetic effect of lowering blood sugar levels.

### Intestine

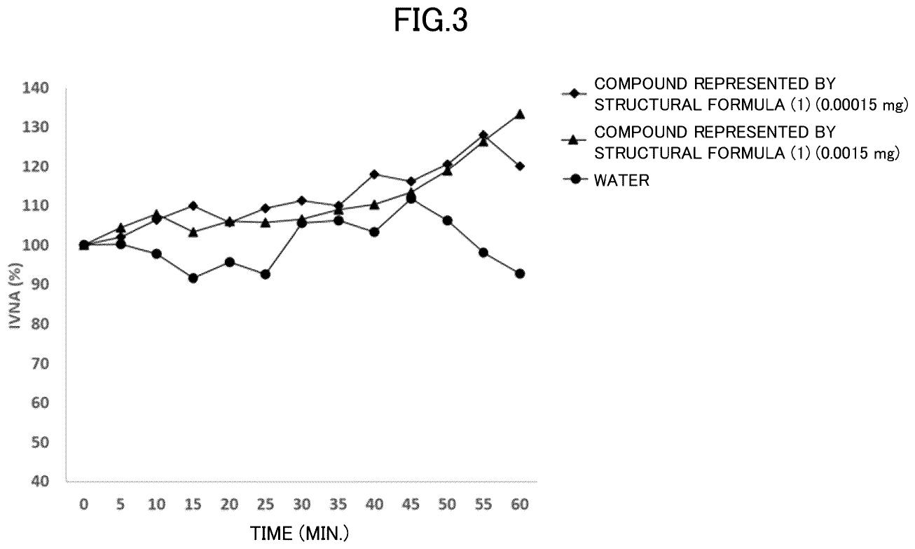

FIG. 3 illustrates the result of the change in the neural activity of the distal branches of vagus (parasympathetic) nerve for the intestine (intestinal vagal nerve activity, IVNA) when the aqueous solution including 0.0015 mg or 0.00015 mg of the compound represented by the structural formula (1) was intragastrically administered at a dose of 0.5 mL/300 g of body weight, or water serving as the solvent was intragastrically administered at a dose of 0.5 mL/300 g of body weight, as a percentage with respect to the mean value (a value at 0 minutes) of 5 minutes before the onset of stimulation being 100%.

As presented in FIG. 3, it was made clear that the group to which the aqueous solution including 0.0015 mg of the compound represented by Structural Formula (1) was administered at the dose of 0.5 mL/300 g of body weight (“ [symbol] ” in FIG. 3) and the group to which the aqueous solution including 0.00015 mg of the compound represented by Structural Formula (1) was administered at the dose of 0.5 mL/300 g of body weight (“♦” in FIG. 3) had the IVNA values higher than the IVNA value of the group to which water serving as the solvent was administered at the dose of 0.5 mL/300 g of body weight (“●” in FIG. 3). It was found from the above results that the compound represented by the structural formula (1) had the effect of activating the activity of the parasympathetic nerve innervating the intestines (small intestine and large intestine).

As the activity level of the parasympathetic nerve innervating the intestine is increased, digestive and adsorptive capability of the intestine is enhanced, and the peristalsis action of the intestine is facilitated, thereby promoting the bowel movement.

### Formulation Example 1

Tablets having the following formulation were prepared according to a common practice.
The compound represented by Structural Formula (1): 5.0 mg
Dolomite: 83.4 mg (including 20% of calcium and 10% of magnesium)
Casein phosphopeptide: 16.7 mg
Vitamin C: 33.4 mg
Maltitol: 136.8 mg
Collagen: 12.7 mg
Sucrose fatty acid ester: 12.0 mg

### Formulation Example 2

An oral liquid preparation having the following formulation was prepared according to a common practice.

### Formulation for one Ampule (100 mL per ampule)

The compound represented by Structural Formula (1): 0.3% by mass
Sorbitol: 12.0% by mass
Sodium benzoate: 0.1% by mass
Fragrance: 1.0% by mass
Calcium sulfate: 0.5% by mass
Purified water: balance

### Formulation Example 3

A coffee drink having the following formulation was prepared according to a common practice.
The compound represented by Structural Formula (1): 0.1% by mass
Coffee extract: 40.0% by mass (L (brightness)=20, Brix=3)
Maltitol: 2.0% by mass
Fragrance: appropriate amount
Water: balance

### Formulation Example 4

Capsules having the following formulation were prepared according to a common practice. As capsules, hard gelatin No. 1 capsules were used.

### Composition in 1 Capsule (200 mg per Capsule)

The compound represented by Structural Formula (1): 30.0 mg
Corn starch: 70.0 mg
Lactose: 80.0 mg
Calcium lactate: 10.0 mg
Hydroxypropyl cellulose (HPC-L): 10.0 mg

The present application claims priority based on Japanese Patent Application No. 2022-079699, filed on May 13, 2022, the entire content of which are incorporated herein by reference.

1. A parasympathetic nerve activation agent comprising:
a compound represented by Structural Formula (1) below,

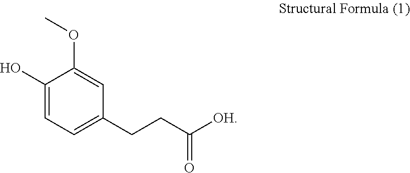

2. The parasympathetic nerve activation agent according to claim 1,
wherein the parasympathetic nerve is a parasympathetic nerve innervating at least one selected from a group consisting of stomach, pancreas, and intestines.

3. A parasympathetic nerve activation composition, comprising:
the parasympathetic nerve activation agent according to claim 1; and
at least one selected from the group consisting of excipients, moisture-proof agents, preservatives, enhancers, thickeners, emulsifiers, antioxidants, sweeteners, acidulants, seasonings, coloring agents, fragrances, whitening agents, humectants, oil components, UV absorbers, surfactants, alcohols, powder components, colorants, aqueous components, water, skin nutrients, and auxiliary agent.

4. A parasympathetic nerve activation composition, comprising:
the parasympathetic nerve activation agent according to claim 2; and
at least one selected from the group consisting of excipients, moisture-proof agents, preservatives, enhancers, thickeners, emulsifiers, antioxidants, sweeteners, acidulants, seasonings, coloring agents, fragrances, whitening agents, humectants, oil components, UV absorbers, surfactants, alcohols, powder components, colorants, aqueous components, water, skin nutrients, and auxiliary agent.
